(19) United States
(12) Patent Application Publication
(10) Pub. No.: US 2011/0003377 A1
(43) Pub. Date: Jan. 6, 2011

(54) SPECIMEN PROCESSING APPARATUS AND SPECIMENT PROCESSING METHOD

(76) Inventor: Teruaki ITOH, Kumamoto-shi (JP)

Correspondence Address: NIXON & VANDERHYE, PC, 901 NORTH GLEBE ROAD, 11TH FLOOR, ARLINGTON, VA, 22203, US

(21) Appl. No.: 12/775,737

(22) Filed: May 7, 2010

(30) Foreign Application Priority Data
Jul. 1, 2009 (JP) 2009-157102

(51) Int. Cl. C12M 1/00

(52) U.S. Cl. 435/307.1

(57) ABSTRACT

According to an example of the invention, a specimen processing apparatus which distributes specimen containers capable of containing a specimen each to a plurality of outlet ports, includes, a plurality of inlet ports, the outlet ports disposed downstream relative to the inlet ports, a plurality of main transport paths which convey the specimen containers from the inlet ports to the outlet ports, a plurality of auxiliary transport paths which diverge from the main transport paths, connect the main transport paths to one another, and convey the specimen containers on the main transport paths to the alternative main transport paths, and a guide unit which guides the transport direction of the specimen containers between the main transport paths and the auxiliary transport paths.

### CROSS-REFERENCE TO RELATED APPLICATIONS

This application is based upon and claims the benefit of priority from prior Japanese Patent Application No. 2009-157102, filed Jul. 1, 2009, the entire contents of which are incorporated herein by reference.

### BACKGROUND OF THE INVENTION

1. Field of the Invention

The present invention relates to a specimen processing apparatus, and more specifically, to an apparatus configured to distribute specimen containers to a plurality of positions.

2. Description of the Related Art

In a specimen processing apparatus configured so that specimens of blood or the like are contained in containers, such as test tubes, as they are conveyed for various processes, the specimen containers are sorted for the conveyance (e.g., Jpn. Pat. Appln. KOKAI Publication No. 2008-76185). In this specimen processing apparatus, identification data is read from barcodes affixed to the respective side surfaces of a plurality of specimen containers that are conveyed on a single transport path. Based on the read identification data, the specimen containers are divergently conveyed downstream to a plurality of transport paths. In the specimen processing apparatus of this type, the transport paths are formed so as to extend continuously from a single inlet port to a plurality of outlet ports. Specifically, the single transport path on the loading side diverges into a plurality of branch transport paths, which are connected to the outlet ports, individually. A gate portion for guiding the transport direction of the specimen containers is disposed at a branch portion.

The specimen containers are distributed to the branch transport paths by the gate portion and conveyed toward the appropriate outlet ports.

However, the above technique has the following problem. Specifically, in the specimen processing apparatus described above, the specimen containers sequentially delivered from the single inlet port are sorted one after another. Thus, sorting the specimen containers takes a long time.

### BRIEF SUMMARY OF THE INVENTION

According to an example of the invention, a specimen processing apparatus which distributes specimen containers capable of containing a specimen each to a plurality of outlet ports, comprises, a plurality of inlet ports, the outlet ports disposed downstream relative to the inlet ports, a plurality of main transport paths which convey the specimen containers from the inlet ports to the outlet ports, a plurality of auxiliary transport paths which diverge from the main transport paths, connect the main transport paths to one another, and convey the specimen containers on the main transport paths to the alternative main transport paths, and a guide unit which guides the transport direction of the specimen containers between the main transport paths and the auxiliary transport paths.

According to another aspect of the invention, the main transport paths are disposed in a predetermined first direction, the auxiliary transport paths are disposed in a second direction perpendicular to the first direction, and the main transport paths and the auxiliary transport paths are arranged in a network.

According to another aspect of the invention, the specimen processing apparatus further comprises a reading unit, which is disposed upstream relative to branch portions at which the auxiliary transport paths diverge from the main transport paths and read labels affixed to the test tubes, thereby acquiring specimen data on the specimens, and a control unit which controls operation of the guide unit based on the data read by the reading unit.

According to another aspect of the invention, the guide unit comprises guide arms which are disposed at branch portions at which the auxiliary transport paths diverge from the main transport paths and guide the specimen containers to the downstream side of the main transport paths or the auxiliary transport paths.

According to another aspect of the invention, the guide unit comprises a transfer unit which transfers the test tubes from the auxiliary transport paths on one side to the auxiliary transport paths on the other side, passing above the main transport paths, at intersections at which the main transport paths and the auxiliary transport paths cross one another.

According to another aspect of the invention, the specimen processing apparatus further comprises a post-processing device which comprises a plurality of transport paths individually continuously extending downstream relative to the outlet ports and post-processing portions which are disposed along the transport paths and process the specimen containers.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a perspective view schematically showing a configuration of a specimen processing apparatus according to an embodiment of the invention;

FIG. 2 is a plan view of the specimen processing apparatus according to the embodiment;

FIG. 3 is a front view of a loading-sorting unit according to the embodiment;

FIG. 4 is a cutaway side view of the loading-sorting unit according to the embodiment;

FIG. 5 is a view illustrating a sorting process according to the embodiment;

FIG. 6 is a view illustrating the sorting process according to the embodiment;

FIG. 7 is a view illustrating the sorting process according to the embodiment;

FIG. 8 is a view illustrating a specimen processing apparatus according to another embodiment of the invention; and

FIG. 9 is a view illustrating a specimen processing apparatus according to still another embodiment of the invention.

### DETAILED DESCRIPTION OF THE INVENTION

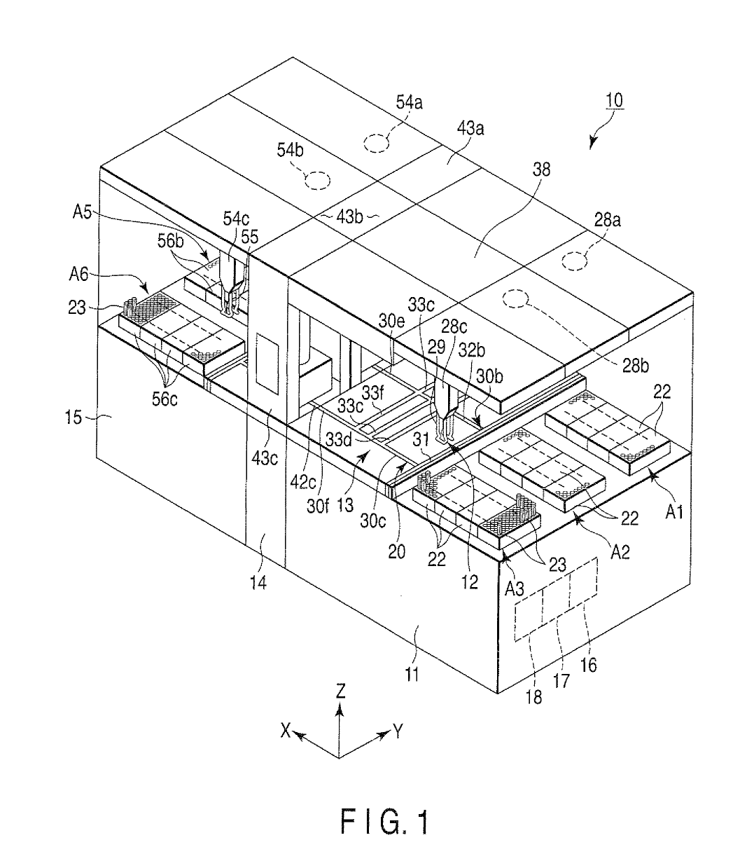

A specimen processing apparatus 10 according to an embodiment of the present invention will now be described with reference to FIGS. 1 to 4. In each of these drawings, configurations are enlarged, reduced, or omitted as required. In each drawing, arrows X, Y and Z indicate three orthogonal directions, individually. In this case, the X-, Y-, and Z-axes extend in longitudinal (left-right), transverse (front-rear), and vertical directions, respectively.

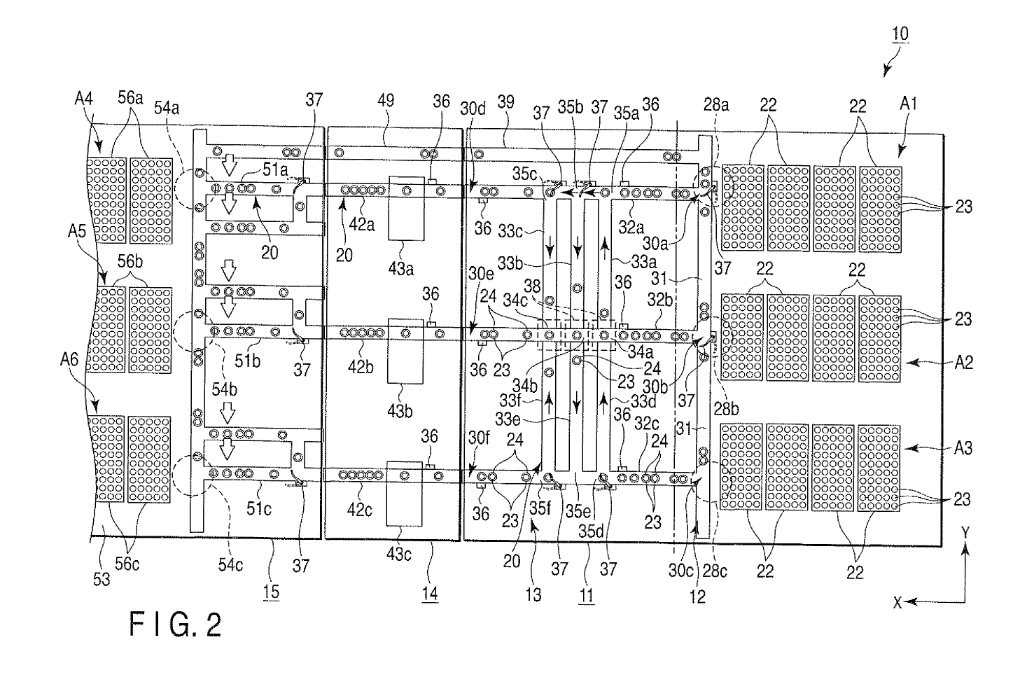

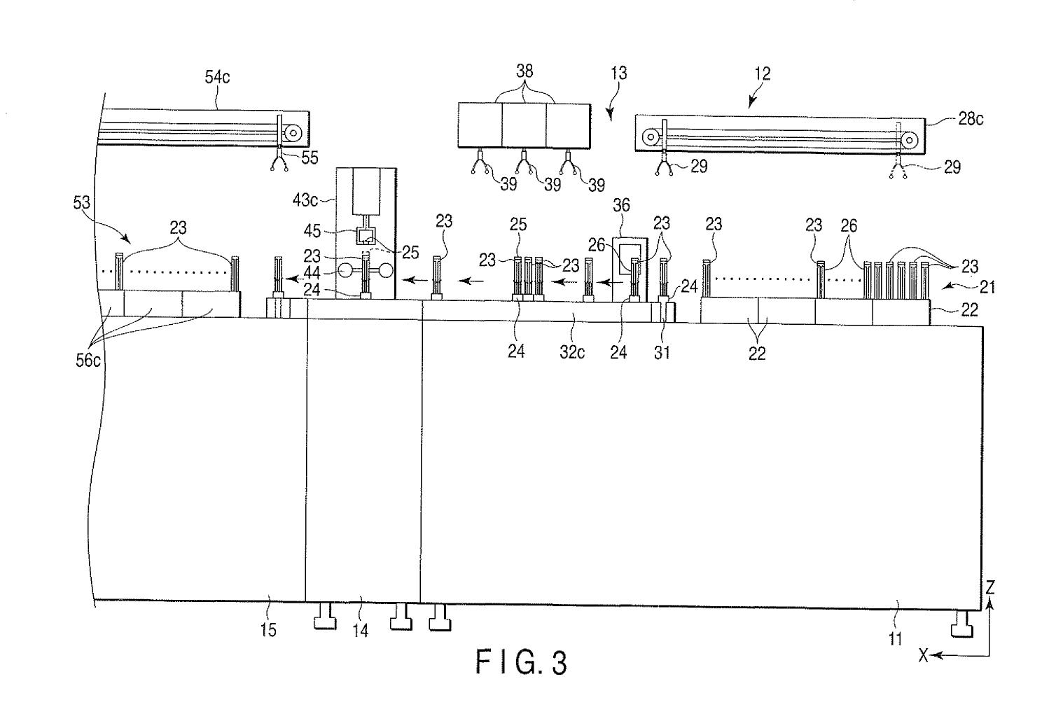

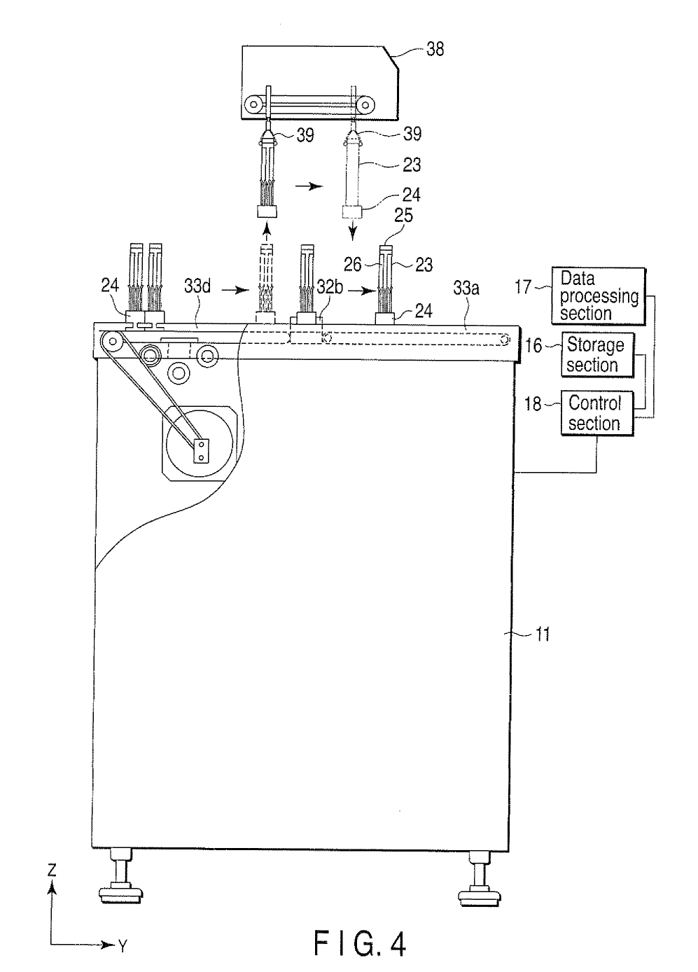

FIGS. 1 and 2 are perspective and plan views, respectively, schematically showing the specimen processing apparatus 10, and FIGS. 3 and 4 are front and side views, respectively, showing a loading-sorting unit 11.

The specimen processing apparatus 10 comprises the loading-sorting unit 11, a cap removal unit 14, and an unloading unit 15, which are arranged side by side. These units 11, 14 and 15 are connected with a storage section (storage unit) 16, data processing section 17, and control section (control unit) 18. The storage section 16 stores various pieces of data. The data processing section 17 performs data processing, such as arithmetic operation and determination, based on identification data. The control section 18 controls operations of the individual sections.

As shown in FIGS. 1 to 3, the loading-sorting unit 11 comprises a loading section (loading unit) 12 and sorting section (sorting unit) 13. The loading section 12 transfers test tubes 23 set in racks 22. The sorting section 13 distributes and guides the loaded test tubes 23 to predetermined routes. The loading section 12 comprises a rack mounting portion 21 and first to third transfer mechanisms 28a to 28c arranged above the rack mounting portion 21.

The test tube racks 22 that each contain a plurality of test tubes 23 are placed and set on the rack mounting portion 21. The racks 22 hold a plurality of rows of standing test tubes 23 in both the left-right and front-rear directions. Each test tube 23 for use as a specimen container is a cylinder that contains blood or the like. A label 26 with a barcode indicative of various pieces of data, such as identification data, on each specimen is affixed to the side surface of each test tube 23.

The first to third transfer mechanisms 28a, to 28c comprises a transfer arm 29 that can hold each test tube 23. Each arms are configured to move transversely, longitudinally, and vertically in predetermined regions corresponding to areas A1 to A3 that are transversely arranged side by side.

A plurality of transfer arms 29 move holding the test tubes 23 set on their corresponding areas of the rack mounting portion 21 and transfer the test tubes to a holder transport portion 20 in the adjacent sorting section 13. Empty holders 24 are previously set on standby on the holder transport portion 20. If the test tubes 23 are transferred to the holders 24 by the transfer arms 29, they are conveyed downstream along a predetermined transport path 30 at the holder transport portion 20.

The sorting section 13 comprises the conveyor-type holder transport portion 20, reading devices 36, guide arms 37 (guide unit), and holder transfer mechanisms 38 (guide unit). The holder transport portion 20 conveys the holders 24 holding the test tubes 23 downstream along the transport path 30, as indicated by arrows in FIG. 3. The reading devices 36 read the labels 26 on the respective side surfaces of the test tubes 23 conveyed on the transport path 30. The guide arms 37 guide the transport direction of the holders 24 under the control of the control section 18. The holder transfer mechanisms 38 hold and transfer the holders 24 under the control of the control section 18. In this embodiment, the test tubes 23 are sorted and delivered to three outlet ports 30d to 30f.

The holder transport portion 20, which is of the conveyor type, comprises a pair of guide rails, a conveyor belt, and conveyor rollers. The guide rails are disposed along the transport path 30 with a fixed space between them. The conveyor belt is located between the guide rails. The conveyor rollers are rotated behind the conveyor belt so as to move the belt. The holders 24 are supported upright on the conveyor belt, each holding one of the test tubes 23, and are conveyed as the conveyor belt travels.

The transport path 30 comprises a loading path 31, first to third inlet ports 30a to 30c, the first to third outlet ports 30d to 30f, first to third main transport paths 32a to 32c, and first to sixth auxiliary transport paths 33a to 331. The loading path 31 extends transversely (in the Y-axis direction) on the loading side at the right of FIG. 2. The inlet ports 30a to 30c are transversely arranged side by side along the loading path 31 on the loading side (upstream side) at the right of FIG. 2. The outlet ports 30d to 30f are transversely arranged side by side on the unloading side (downstream side) at the left of FIG. 2. The main transport paths 32a to 32c extend longitudinally (in the X-axis direction) in parallel relation and connect the inlet ports 30a to 30c to the outlet ports 30d to 30f, respectively. The auxiliary transport paths 33a to 331 diverge from the main transport paths 32a to 32c so as to extend transversely in parallel relation and connect the adjacent main transport paths 32a to 32c.

The first to third main transport paths 32a to 32c convey the holders 24 longitudinally (in the X-axis direction) from right to left in FIG. 2.

The first auxiliary transport path 33a diverges from the second main transport path 32b and conveys the holders 24 rearward (or toward the positive end of the Y-axis) toward the first main transport path 32a. The second auxiliary transport path 33b diverges from the first main transport path 32a and conveys the holders 24 forward (or toward the zero end of the Y-axis) toward the second main transport path 32b. The third auxiliary transport path 33c diverges from the first main transport path 32a and conveys the holders 24 transversely forward toward the second main transport path 32b. The fourth auxiliary transport path 33d conveys the holders 24 transversely rearward from the third main transport path 32c toward the second main transport path 32b. The fifth auxiliary transport path 33e conveys the holders 24 transversely forward from the second main transport path 32b toward the third main transport path 32c. The sixth auxiliary transport path 33f conveys the holders 24 transversely rearward from the third main transport path 32c toward the second main transport path 32b.

The first and fourth auxiliary transport paths 33a and 33d are located in the same position with respect to the X-direction and transversely arranged with the second main transport path 32b between them. The second and fifth auxiliary transport paths 33b and 33e are located in the same position with respect to the X-direction and transversely arranged with the second main transport path 32b between them. The third and sixth auxiliary transport paths 33c and 33f are located in the same position with respect to the X-direction and transversely arranged with the second main transport path 32b between them.

The three or first to third main transport paths 32a to 32c, which extend longitudinally, and the six auxiliary transport paths 33a to 33f, which extend transversely, are arranged crossing one another in a network. Three intersections 34a to 34c are formed on the second main transport path 32b. Three branch portions 35a to 35c are formed on the first main transport path 32a, and another three branch portions 35d to 35f on the third main transport path 32c.

The reading devices 36 are individually disposed on the upstream side of the branch portions 35a to 35f and intersections 34a to 34c. The reading devices 36 are arranged beside the transport path 30 and serve to optically read the labels 26 on the respective side surfaces of the test tubes 23 fed by the holder transport portion 20, thereby acquiring identification data, such as barcode data, on the specimens. The data acquired by the reading devices 36 is stored in the storage section 16 and used for the control of switching operations of the guide arms 37 and transfer operations of the holder transfer mechanisms 38.

The guide arms 37 are disposed individually at the branch portions 35b and 35c on the first main transport path 32a, the branch portions 35d and 35f on the third main transport path 32c, and the inlet ports 30a and 30b. By pivoting at the branch portions under the control of the control section 18, the guide arms 37 guide the transport direction of the holders 24 for the transport paths that diverge downstream. The transport direction of the holders 24 is settled based on the specimen data acquired by the reading devices 36.

The holder transfer mechanisms 38 are disposed individually on the two intersections 34a and 34b on the second main transport path 32b. As shown in FIG. 4, each holder transfer mechanism 38 comprises a transfer arm 39 capable of holding each test tube 23. Under the control of the control section 18, the holders 24 on one side of the second main transport path 32b are held individually by the transfer arms 39. The holders 24 are transversely transferred to the auxiliary transport paths on the other side, passing above the holders 24 and test tubes 23 conveyed longitudinally on the second main transport path 32b. As described later, for example, the holders 24 are transferred from the fourth auxiliary transport path 33d to the first auxiliary transport path 33a, passing above the second main transport path 32b. Further, the holders 24 are transferred from the second auxiliary transport path 33b to the fifth auxiliary transport path 33e, passing above the second main transport path 32b.

The cap removal unit 14 for use as a post-processing device comprises the holder transport portion 20 and first to third cap removal portions 43a to 43c. The holder transport portion 20 comprises transport paths 42a to 42c that are continuous with the first to third main transport paths 32a to 32c, respectively. The three cap removal portions 43a to 43c are located beside the transport paths 42a to 42c, respectively. Each of the cap removal portions 43a to 43c holds each test tube 23 by means of its clamp mechanism 44 as it seizes and raises a cap 25 by means of its holding mechanism 45. By doing this, a cap removal process is performed in which the cap 25 fitted in a top opening of the test tube 23 is removed.

The unloading unit 15 for use as a post-processing device comprises the holder transport portion 20, a rack mounting portion 53, and transfer mechanisms 54a to 54c. The holder transport portion 20 comprises transport paths 51a to 51c that are connected to the transport paths 42a to 42c, respectively. The transfer mechanisms 54a to 54c are transversely arranged side by side above the rack mounting portion 53. The rack mounting portion 53 is divided into a plurality of areas A4 to A6 that are transversely arranged side by side. A plurality of test tube racks 56a to 56c are placed and set on areas A4 to A6, respectively.

The transfer mechanisms 54a to 54c are configured to move transversely, longitudinally, and vertically in predetermined regions corresponding to areas A4 to A6, individually. Each transfer mechanism comprises a transfer arm 55 that can hold each test tube 23. The transfer arms 55 hold the test tubes 23 on the transport paths 51a to 51c and transfer them to the racks 56a to 56c in corresponding areas A4 to A6.

The following is a description of processing procedure of the specimen processing apparatus 10 according to the present embodiment.

First, in the loading section 12, a loading process is performed in which the test tubes 23 previously set in the racks 22 are sequentially transferred to the loading section 12. Since this process precedes a sorting process, the holders to which the test tubes 23 are transferred do not need to be specified. For example, the test tubes 23 are transferred to the holders 24 set near areas A1 to A3. Since the three transfer mechanisms 28a to 28c simultaneously load the test tubes 23, the processing time can be reduced.

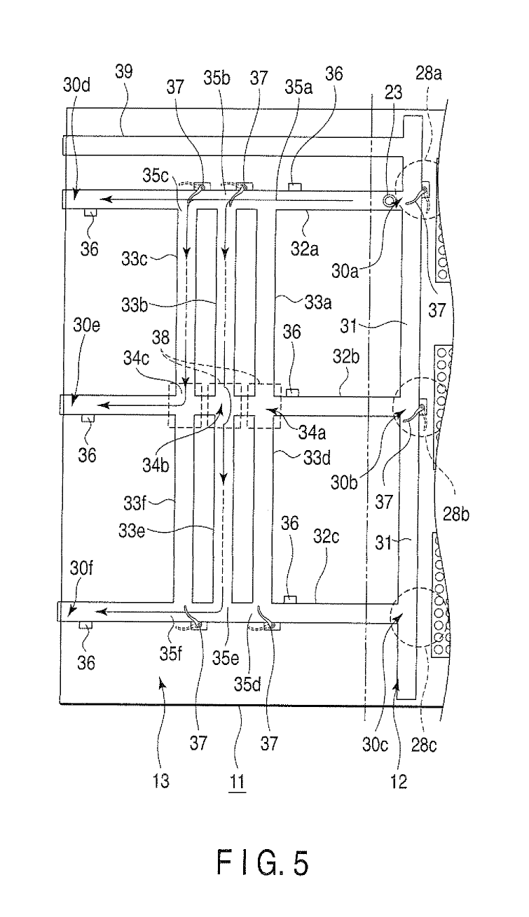

Then, the sorting process is performed in the sorting section 13. The procedure of the sorting process will now be described with reference to FIGS. 5 to 7. First, the test tubes 23 are conveyed downstream along the first to third main transport paths 32a to 32c from the corresponding inlet ports 30a to 30c. During this conveyance, a reading process is performed in which the labels 26 on the respective side surfaces of the test tubes 23 are optically read by the reading devices 36 that are disposed short of the branch portions 35b and 35c and intersections 34a to 34c, whereby identification data, such as barcode data, on the specimens is acquired.

Based on the read specimen data, the guide and transfer operations of the guide arms 37 and transfer mechanisms 38 are performed under the control of the control section 18 as the branch portions 35b and 35c and intersections 34a to 34c are passed. The sorting process is performed by the conveying motion of the transport portion 20 that moves the test tubes 23 along the transport path 30 and the guide and transfer operations of the guide arms 37 and transfer mechanisms 38 controlled by the control section 18. In this sorting process, the test tubes 23 are sorted along predetermined routes shown in FIGS. 5 to 7 and delivered to the specific outlet ports 30d to 30f.

FIG. 5 shows the sorting process for the test tubes from the first inlet port 30a. Each test tube 23 determined to be conveyed to, for example, the first outlet port 30d is guided straight to the left in FIG. 5 on the first main transport path 32a and conveyed to the first outlet port 30d.

Each test tube 23 determined to be conveyed to the second outlet port 30e is guided onto the third auxiliary transport path 33c by the guide arm 37 at the branch portion 35c on the first main transport path 32a. Then, by the conveying motion of the path 33c, the test tube 23 is delivered to the second main transport path 32b, which underlies the path 33c as in FIG. 5, and moves onto the path 32b at the intersection 34c. By the conveying motion of the path 32b, moreover, the test tube 23 is fed to the left and guided to the second outlet port 30e.

Each test tube 23 determined to be conveyed to the third outlet port 301 is guided onto the second auxiliary transport path 33b by the guide arm 37 at the branch portion 35b on the first main transport path 32a. Then, by the conveying motion of the path 33b, the test tube 23 is fed forward (or downward in FIG. 5) and transferred to the fifth auxiliary transport path 33e by the holder transfer mechanism 38 at the intersection 34b, passing above the second main transport path 32b. By the conveying motion of the path 33e, moreover, the test tube 23 is fed forward to the branch portion 35e, from which it moves onto the third auxiliary transport path 33c. Then, the test tube 23 is guided leftward to the third outlet port 30f by the conveying motion of the path 33c.

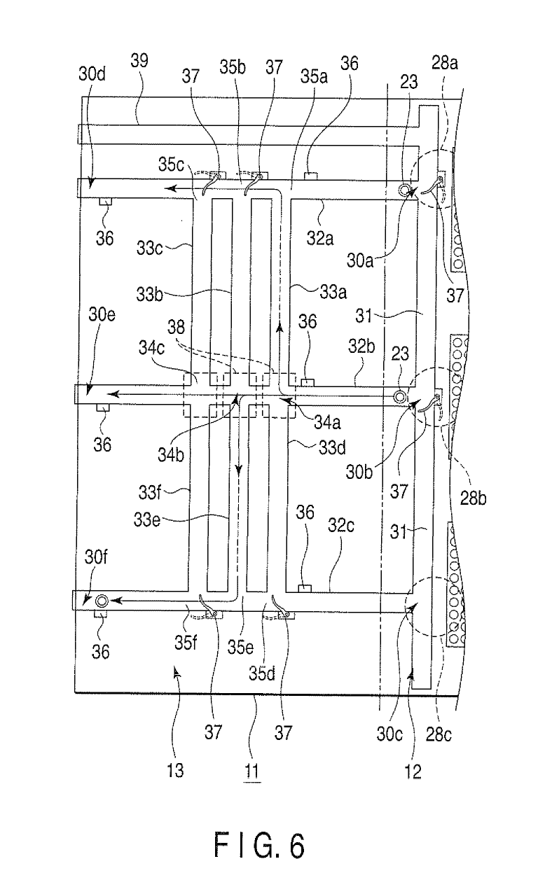

FIG. 6 shows the sorting process for the test tubes from the second inlet port 30b. Each test tube 23 determined to be conveyed to, for example, the first outlet port 30d is transferred to the first auxiliary transport path 33a by the holder transfer mechanism 38 at the intersection 34a on the second main transport path 32b. Then, by the conveying motion of the path 33a, the test tube 23 is fed rearward (or upward in FIG. 6) and moves to the branch portion 35a on the first main transport path 32a. By the conveying motion of the path 32a, moreover, the test tube 23 is fed to the left and conveyed to the first outlet port 30d.

Each test tube 23 determined to be conveyed to the second outlet port 30e advances straight to the left on the second main transport path 32b and is delivered to the second outlet port 30e.

Each test tube 23 determined to be conveyed to the third outlet port 30f is transferred to the fifth auxiliary transport path 33e by the holder transfer mechanism 38 at the intersection 34b on the second main transport path 32b. Then, the test tube 23 advances forward on the fifth auxiliary transport path 33e and is delivered to the third main transport path 32c. Further, the test tube 23 is guided leftward on the path 32c from the branch portion 35e to the third outlet port 30f.

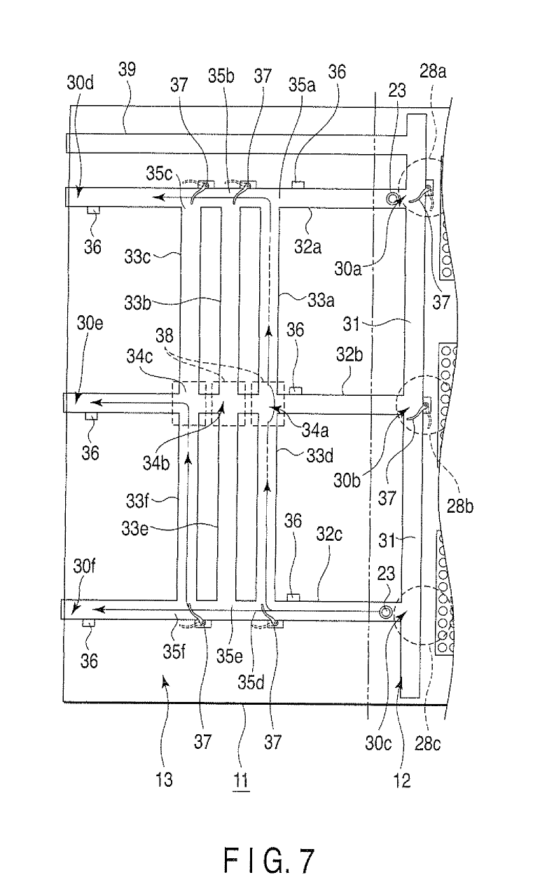

FIG. 7 shows the sorting process for the test tubes from the third inlet port 30c. Each test tube 23 determined to be conveyed to, for example, the first outlet port 30d is guided rearward to the fourth auxiliary transport path 33d by the guide arm 37 at the branch portion 35d on the third main transport path 32c. Then, the test tube 23 moves rearward on the fourth auxiliary transport path 33d toward the intersection 34a. At the intersection 34a, the test tube 23 is transferred to the first auxiliary transport path 33a by the holder transfer mechanism 38. Then, the test tube 23 is fed rearward (or upward in FIG. 7) by the path 33a and moves to the branch portion 35a on the first main transport path 32a. Further, the test tube 23 is fed to the left by the path 32a and guided to the first outlet port 30d.

Each test tube 23 determined to be conveyed to the second outlet port 30e is guided upward to the sixth auxiliary transport path 33f by the guide arm 37 at the branch portion 35f on the third main transport path 32c. Then, the test tube 23 is fed rearward to the intersection 34c by the path 33f and moves to the second main transport path 32b. Further, the test tube 23 is fed leftward to the second outlet port 30e by the path 32b.

Each test tube 23 determined to be conveyed to the third outlet port 301 moves straight to the left along the third auxiliary transport path 33c and advances to the third outlet port 30f.

Thus, a large number of test tubes 23 that are fed at random from the inlet ports 30a to 30c are sorted and conveyed to the outlet ports 30d to 30f that are specified based on the specimen data. The processing time can be reduced by simultaneously processing the test tubes 23 by means of the three main transport paths 32a to 32c and six auxiliary transport paths 33a to 331 that are arranged in a network.

The test tubes 23 distributed to the three outlet ports 30d to 30f are delivered downstream to the three transport paths 42a to 42c of the cap removal unit 14 that are connected to the outlet ports 30d to 301, respectively.

The cap removal process for removing the caps 25 from the test tubes 23 is performed in the cap removal unit 14. First, specimen data on each test tube 23 is acquired as the test tube passes through the reading device 36 on the upstream side, and the necessity of cap removal is determined by the acquired data. Based on the result of the determination, the caps 25 are removed from only those test tubes 23 which need to be uncapped. Each of the cap removal portions 43a to 43c holds each test tube 23 by means of the clamp mechanism 44 as it seizes and raises the cap 25 by means of the holding mechanism 45. By doing this, the cap removal process is performed to remove the cap 25 fitted in the top opening of the test tube 23. The uncapped test tubes 23 and those test tubes 23 which do not need be uncapped are fed downstream along the transport paths to the unloading unit 15.

Then, in the unloading unit 15, an unloading process is performed in which the test tubes 23 in the holder transport portion 20 are held by the transfer arms 55 and transferred to the specified racks 56a to 56c. The test tubes distributed to the outlet port 30d are loaded into the rack 56a through the transport paths 42a and 51a. The test tubes distributed to the outlet port 30e are loaded into the rack 56b through the transport paths 42b and 51b. The test tubes distributed to the outlet port 301 are loaded into the rack 56c through the transport paths 42c and 51c.

In this manner, the test tubes 23 are distributed to areas A4 to A6 corresponding to the outlet ports 30d to 301, respectively. This unloading operation is also simultaneously performed at three positions, so that the processing time can be reduced.

The specimen processing apparatus 10 according to the present embodiment can provide the following effects. Specifically, the processing apparatus 10 that distributes the test tubes 23 to the outlet ports 30d to 30f are provided with the inlet ports 30a to 30c, main transport paths 32a to 32c, and auxiliary transport paths 33a to 331 that connect the main transport paths 32a to 32c to one another. Thus, a plurality of test tubes 23 can be simultaneously processed, so that the processing time can be reduced. In this case, the various processes are simultaneously performed at three positions. In sorting a large number of test tubes 23, therefore, the processing time can be reduced to about ⅓. Since the transport paths are connected in a common network for the simultaneous performance of the processes, moreover, the space can be saved.

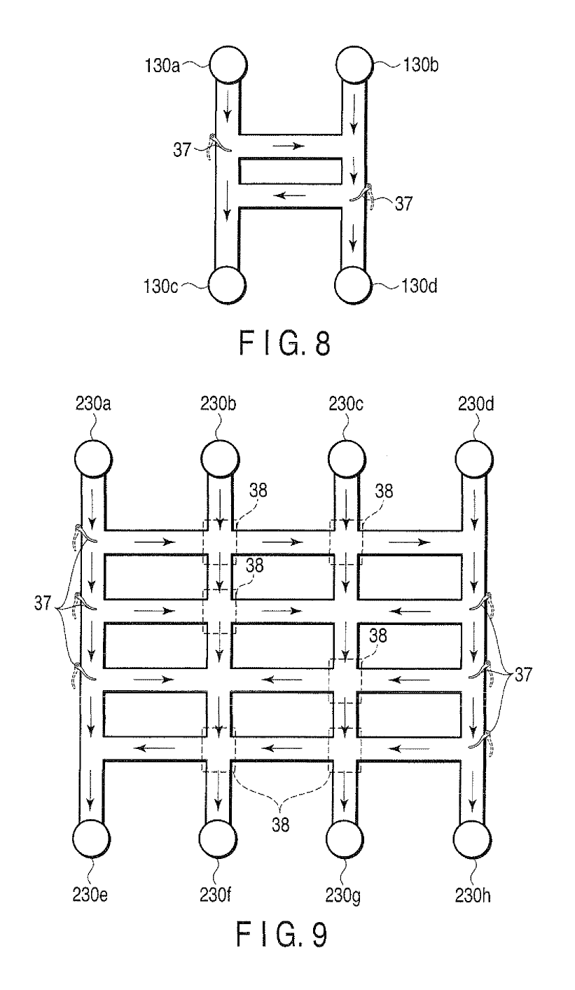

The present invention is not limited directly to the embodiment described above, and its constituent elements may be embodied in modified forms without departing from the scope or spirit of the invention. In the above embodiment, the inlet ports, outlet ports, and main transport paths are arranged in three pairs. However, the same effects can be obtained from, for example, an alternative specimen processing apparatus 100, which comprises inlet ports 130a and 130b and outlet ports 130c and 130d arranged in two parallel pairs, as shown in FIG. 8, or another specimen processing apparatus 200, which comprises inlet ports 230a to 230d and outlet ports 230e to 230h arranged in four parallel pairs, as shown in FIG. 9. Further, the mechanisms that transfer the test tubes 23 or holders 24 one after another may be replaced, as the transfer unit, with mechanisms that transfer the test tubes or holders collectively. Furthermore, the specimen processing apparatus 10 according to the above embodiment comprises the loading-sorting unit 11, cap removal unit 14, and unloading unit 15 that are arranged continuously. Alternatively, the cap removal unit 14 may be omitted or another processing unit may be added, for example.

Further, some of the constituent elements according to the above embodiment may be omitted, and the shapes, structures, materials, etc., of the constituent elements may be changed. Furthermore, various inventions can be formed by appropriately combining the constituent elements disclosed in the above embodiment.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A specimen processing apparatus which distributes specimen containers capable of containing a specimen each to a plurality of outlet ports, comprising:
a plurality of inlet ports;
the outlet ports disposed downstream relative to the inlet ports;
a plurality of main transport paths which convey the specimen containers from the inlet ports to the outlet ports;
a plurality of auxiliary transport paths which diverge from the main transport paths, connect the main transport paths to one another, and convey the specimen containers on the main transport paths to the alternative main transport paths; and
a guide unit which guides the transport direction of the specimen containers between the main transport paths and the auxiliary transport paths.

2. A specimen processing apparatus according to claim 1, wherein the main transport paths are disposed in a predetermined first direction, the auxiliary transport paths are disposed in a second direction crossing the first direction, and the main transport paths and the auxiliary transport paths are arranged in a network.

3. A specimen processing apparatus according claim 1, further comprising a reading unit, which is disposed upstream relative to branch portions at which the auxiliary transport paths diverge from the main transport paths and read labels affixed to the test tubes, thereby acquiring specimen data on the specimens, and a control unit which controls operation of the guide unit based on the data read by the reading unit.

4. A specimen processing apparatus according to claim 1, wherein the guide unit comprises guide arms which are disposed at branch portions at which the auxiliary transport paths diverge from the main transport paths and guide the specimen containers to the downstream side of the main transport paths or the auxiliary transport paths by pivoting.

5. A specimen processing apparatus according to claim 1, wherein the guide unit comprises a transfer unit which transfers the test tubes from the auxiliary transport paths on one side to the auxiliary transport paths on the other side, passing above the main transport paths, at intersections at which the main transport paths and the auxiliary transport paths cross one another.

6. A specimen processing apparatus according to claim 1, further comprising a post-processing device which comprises a plurality of transport paths individually continuously extending downstream relative to the outlet ports and post-processing portions which are disposed along the transport paths and process the specimen containers.
